(19) United States
(12) Patent Application Publication
(10) Pub. No.: US 2006/0171970 A1
(43) Pub. Date: Aug. 3, 2006

(54) Pharmaceutical formulation delivery system

(75) Inventor: Mitchell S. Karl, Boca Raton, FL (US)

Correspondence Address: RUDEN, MCCLOSKY, SMITH, SCHUSTER & RUSSELL, P.A., 222 LAKEVIEW AVE, SUITE 800, WEST PALM BEACH, FL, 33401-6112, US

(73) Assignee: Cardio Combos LLC

(21) Appl. No.: 11/365,563

(22) Filed: Mar. 1, 2006

Related U.S. Application Data
(63) Continuation-in-part of application No. 10/785,169, filed on Feb. 23, 2004.
(60) Provisional application No. 60/449,470, filed on Feb. 21, 2003.

(51) Int. Cl. A61K 9/00; B65D 83/04

(52) U.S. Cl. 424/400; 206/531

(57) ABSTRACT

The invention relates to the development of a simple and inexpensive oral delivery system for customized pharmaceutical formulations for treating patients having conditions such as HIV/AIDS, cancer, and heart disease. The system includes a) a plurality of prepackaged pharmaceutical formulations containing at least two pharmacologically active ingredients in periodic (e.g., daily, twice-daily, three times a day, four times a day) dosage amounts for treating a plurality of patients having differing characteristics and particular conditions to be treated, each formulation differing in pharmacologically active ingredients and/or dosage amounts from each other, and b) a chart listing the plurality of formulations for assisting a physician in deciding which formulation to administer to a patient suffering from a particular condition based on the patient's characteristics. A customized pharmaceutical formulation that is suitable for a patient having a particular condition to be treated is easily selected by the physician from a paper or electronic chart listing a plurality of different pharmaceutical formulations and correlating the different formulations to different patient characteristics that should be considered before administering a pharmaceutical formulation to the patient.

### CROSS-REFERENCE TO RELATED APPLICATION

The present application is a continuation-in-part of U.S. patent application Ser. No. 10/785,169, filed Feb. 23, 2004, which claims the priority of U.S. provisional patent application No. 60/449,470, filed Feb. 21, 2003.

### FIELD OF THE INVENTION

The invention relates generally to the fields of medicine and pharmacology. More particularly, the present invention relates to a pharmaceutical formulation delivery system for oral drug delivery.

### BACKGROUND OF THE INVENTION

A number of issues exist that contribute to a patient's non-compliance with a drug regimen. Patients frequently complain about the cost of each of their medications and look for substitutes for particularly costly ones in their regimen. Patients often run out of, forget to take some of, and have difficulty swallowing medications. For example, HIV patients often find it difficult to swallow pills due to secondary conditions such as esophageal disease (e.g., oropharyngeal candidiasis), esophagitis or non-specific mucositis. Esophageal symptoms commonly experienced by HIV patients include odynophagia (painful swallowing) and dysphagia, a feeling of food sticking in the retrostemal area. Patients may question the validity of a multiple drug regimen and/or feel overmedicated by the number of pills they must swallow.

Physicians' acceptance of combination pill therapy has been limited by heretofor very restricted and limited dosing combinations that do not allow for patient factors that might necessitate dose adjustments. Because of the inadequate available variations found with combination pill therapy, physicians view combination pill therapies as having limited utility in clinical practice. Thus, there is still a need in the art for simple and inexpensive oral delivery systems for drug compositions that are easily prepared, easily ingested, and that can deliver a broad range of pharmacologically active agents.

### SUMMARY OF THE INVENTION

The invention relates to the development of a simple and inexpensive oral delivery system for customized pharmaceutical formulations for treating patients having conditions such as HIV/AIDS, cancer, and heart disease. The system includes a) a plurality of prepackaged pharmaceutical formulations containing at least two pharmacologically active ingredients in periodic (e.g., daily, twice-daily, three times a day, four times a day) dosage amounts for treating a plurality of patients having differing characteristics and particular conditions to be treated, each formulation differing in pharmacologically active ingredients and/or dosage amounts from each other, and b) a chart listing the plurality of formulations for assisting a physician in deciding which formulation to administer to a patient suffering from a particular condition based on the patient's characteristics. A customized pharmaceutical formulation that is suitable for a patient having a particular condition to be treated is easily selected by the physician from a paper or electronic chart listing a plurality of different pharmaceutical formulations and correlating the different formulations to different patient characteristics that should be considered before administering a pharmaceutical formulation to the patient. For example, some patients having congestive heart failure also have impaired renal function, impaired hepatic function, conduction system disease, diabetes, relative hypotension, bradycardia, or an electrolyte abnormality. Thus, the choice of pharmaceutical formulation for a particular patient having congestive heart failure depends on whether or not the patient has one (or more) of the above conditions. Because the system provides a plurality of customized pharmaceutical formulations (containing different pharmacologically active ingredients in varying dosage amounts) to choose from and a simple chart listing a variety of patient characteristics and corresponding pharmaceutical formulations, the system enables the physician to select the most suitable combination and dosing for the patient, while affording the patient greater simplicity, decreased cost, easier compliance, and greater confidence associated with prepackaged periodic (e.g., daily, twice-daily, three times a day, four times a day) dosing.

The pharmaceutical formulations of the invention are preferably provided in liquid form, a form that is easily consumed by patients who have difficulty swallowing pills. They may also be in solid (e.g., powder) form, as well as a combination of liquid and powder that is mixed together just before ingesting. The pharmaceutical formulations described herein can be packaged as periodic (e.g., daily, twice-daily, three times a day, four times a day) dosage units in a variety of ways. For example, packaging for a liquid or a solid (e.g., powder) pharmaceutical formulation can take the packaging form of a plastic (e.g., disposable) receptacle (e.g., dispenser) with a twist-off cap. When the pharmaceutical formulation is a dry powder that is combined with a liquid immediately before ingesting, the packaging can take the form of a plastic receptacle (e.g., dispenser) having a first compartment (e.g., dry plastic reservoir) for containing the dry powder and a second compartment for containing the liquid, the two compartments separated from one another by a rupturable seal or barrier, such that rupturing of the seal or barrier (e.g., by squeezing, twisting, etc.) causes the powder and the liquid to mix. As another example, a blister pack can be used for packaging pharmaceutical formulations in powder form, allowing for easy dispensing of the formulation from the blister pack into the patient's drink of choice.

Accordingly, the invention features a pharmaceutical formulation delivery system. The system includes a) a plurality of prepackaged pharmaceutical formulations each including at least two pharmacologically active ingredients in periodic dosage amounts for treating a condition, each pharmaceutical formulation differing in active ingredients or dosage amounts from each other; and b) a chart listing at least a first set of patient characteristics relating to the condition and a second set of patient characteristics relating to the condition, the chart indicating a pharmaceutical formulation of the plurality of pharmaceutical formulations that is suitable for treating a patient having the first set or second set of patient characteristics. At least one pharmaceutical formulation of the plurality of pharmaceutical formulations includes at least three pharmacologically active ingredients in periodic dosage amounts for treating a condition. The prepackaged pharmaceutical formulations can be in liquid form and prepackaged in a plastic receptacle for containing a liquid, the receptacle including a twist-off cap. At least one pharmaceutical formulation of the plurality of prepackaged pharmaceutical formulations can include a powder and a liquid and be prepackaged in a plastic receptacle, the plastic receptacle including a twist-off cap, a first compartment for containing the powder, and a second compartment for containing the liquid, the first and second compartments separated by a rupturable seal such that rupturing of the seal exposes the powder to the liquid. At least one pharmaceutical formulation of the plurality of pharmaceutical formulations can be in powder form and prepackaged in a sufficient amount to provide dosing for seven days, the at least one pharmaceutical formulation prepackaged in a blister pack including a plurality of deformable blisters therein, each deformable blister containing one day's dose of the at least one pharmaceutical formulation. At least one pharmaceutical formulation of the plurality of pharmaceutical formulations can be in powder form and include at least three pharmacologically active ingredients, the at least one prepackaged pharmaceutical formulation prepackaged in a blister pack including a plurality of deformable blisters therein, each deformable blister containing one pharmacologically active ingredient. At least one pharmaceutical formulation of the plurality of prepackaged pharmaceutical formulations can include a powder and a liquid and be prepackaged in a plastic receptacle, the plastic receptacle including a twist-off cap, at least a first and a second compartment for containing the powder, and a third compartment for containing the liquid, the at least first and second compartments separated from the third compartment by rupturable seals such that rupturing of the seals exposes the powder to the liquid.

In another aspect, the invention features a method of treating a patient having a particular condition. The method includes the steps of: a) evaluating the patient and determining a set of patient characteristics relating to the condition; b) selecting a prepackaged pharmaceutical formulation from a chart listing a plurality of sets of patient characteristics relating to the condition and a plurality of prepackaged pharmaceutical formulations, each pharmaceutical formulation including at least two pharmacologically active ingredients in periodic dosage amounts for treating the condition, the chart indicating which of the plurality of pharmaceutical formulations is suitable for treating the patient; and c) administering the prepackaged pharmaceutical formulation to the patient. The condition can be HIV/AIDS, cancer, heart failure, or coronary artery disease.

Unless otherwise defined, all technical terms used herein have the same meaning as commonly understood by one of ordinary skill in the art to which this invention belongs.

By the term “blister pack” is meant a housing containing a plurality of deformable blisters therein, each blister adapted to accommodate a pharmaceutical formulation. Typically, each blister is a substantially annular disk made from a plastic material having a raised portion defined on one side of the disk and a cavity on an opposite side of the disk corresponding with the raised portion, the raised portion defining the blister and a rupturable seal coupled to the side of the disk having the cavity, by which the cavity is covered. The rupturable seal is typically a frangible foil that hermetically seals the contents within the cavity.

Although compositions, systems, and methods similar or equivalent to those described herein can be used in the practice or testing of the present invention, suitable compositions, systems, and methods are described below. The particular embodiments discussed below are illustrative only and not intended to be limiting.

### BRIEF DESCRIPTION OF THE DRAWINGS

FIG. 1 is a front view of a first embodiment of the invention.

FIG. 2 is a front view and a side view of a second embodiment of the invention.

FIG. 3 is a front view and a side view of a third embodiment of the invention.

FIG. 4 is a perspective view of a fourth embodiment of the invention.

FIG. 5A is a top view of a fifth embodiment of the invention.

FIG. 5B is a top view of a sixth embodiment of the invention.

### DETAILED DESCRIPTION

An exemplary system of the invention includes: a) a plurality of prepackaged pharmaceutical formulations each having at least two pharmacologically active ingredients in periodic (e.g., daily, twice-daily, three times a day, four times a day) dosage amounts for treating a particular condition, and b) a chart listing the pharmacologically active ingredients and dosage amounts of each formulation and correlating the different formulations with different sets of patient characteristics for assisting a physician in selecting the appropriate formulation based on a particular patient's condition and characteristics. A wide variety of pharmacologically active ingredient (e.g., drug) combinations are envisioned for incorporation in the pharmaceutical formulations described herein and are chosen based on the condition to be treated and the characteristics of the patient being treated. Each pharmaceutical formulation of the plurality of formulations differs from every other pharmaceutical formulation of the plurality of formulations with respect to the pharmacologically active ingredients contained therein and/or the dosage amounts of the pharmacologically active ingredients contained therein. For example, a first pharmaceutical formulation may contain pharmacologically active ingredients A and B at dosage amounts of 25 mg and 50 mg, respectively, while a second pharmaceutical formulation may contain pharmacologically active ingredients A and B at dosage amounts of 30 mg and 55 mg, respectively. As another example, a first pharmaceutical formulation may contain pharmacologically active ingredients A, B, and C, while a second pharmaceutical formulation may contain pharmacologically active ingredients A and C.

In some cases, a patient's needs may not exactly match an offered formulation. In such cases, a formulation that very closely fits the patient's needs could be selected from the chart as most suitable and could then be administered to the patient in conjunction with one or two pills that complete the drug regimen. Such a regimen is particularly useful for a patient who is taking at least one medication that is most effective when taken twice a day. In such an instance, one dose of the twice-daily medication is provided in the pharmaceutical formulation ingested by the patient at a first time of day (e.g., morning), while the second dose is ingested by the patient at a second time of day (e.g., evening) in pill or tablet form.

Pharmaceutical formulations described herein are preferably in liquid dosage forms for oral administration, such dosage forms including pharmaceutically acceptable emulsions, solutions, suspensions, syrups, and elixirs containing inert diluents commonly used in the art, such as water. Besides such inert diluents, compositions can also include adjuvants, wetting agents, emulsifying and suspending agents, and sweetening, flavoring, and perfuming agents.

The form (e.g., powder, liquid, suspension) of a pharmaceutical formulation described herein is based on the characteristics (e.g., solubility) of the pharmacologically active ingredients (e.g., drugs) within the formulation. For example, pharmaceutical formulations containing soluble drugs are typically solutions that are flavored and sweetened. One example of a solvent is a combination of water and glycerine, propylene glycol, or polyethylene glycol 400. The pH of such a solution can be adjusted with a buffering agent to be favorable for drug stability. Pharmaceutical formulations containing insoluble drugs can take the form of a suspension. Such a suspension can be made viscous by the use of a suspending agent, e.g., methylcellulose, hydroxyethylcellulose, hydroxypropycellulose, and/or a natural gum (e.g., xanthum gum). For combinations of pharmacologically active ingredients that are not suitable for being made into a stable, palatable, liquid mix (e.g., those containing aspirin), a powder or granular mix of the pharmacologically active ingredients can be formulated and prepackaged in a unit dose for dispensing to be later dispersed with water or other vehicle (e.g., flavored solution). For example, a powder mix of drugs, flavors and sweeteners packaged as a unit dose are dispersed with water immediately prior to consumption by the patient. Such powder or granular mixtures can be mixed with pharmaceutically acceptable carriers or excipients. A description of exemplary pharmaceutically acceptable carriers and excipients, as well as pharmaceutical formulations, can be found in Remington's Pharmaceutical Sciences (20^{th} edition, Mack Publishing Co., PA, 2000), a standard text in this field.

Pharmaceutical formulations can take the form of aqueous suspensions. Those that are aqueous suspensions typically contain the active ingredients in admixture with excipients suitable for the manufacture of aqueous suspensions. The aqueous suspensions may also contain one or more preservatives, for example, ethyl or n-propyl p-hydroxybenzoate; one or more coloring agents; one or more flavoring agents; and one or more sweetening agents such as sucrose or saccharin. In some cases, a pharmaceutical formulation can contain an osmotic-adjusting agent such as sodium chloride, dextrose, sodium bicarbonate, calcium chloride, potassium chloride, sodium lactate, Ringer's solution and lactated Ringer's solution.

Oily suspensions may be formulated by suspending the active ingredients in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents and flavoring agents may be added to provide a palatable oral preparation.

Pharmaceutical formulations of the invention may also be in the form of oil-in-water emulsions. The oily phase may be a vegatable oil such as olive oil or arachis oil, or a mineral oil such as liquid paraffin or a mixture thereof. Suitable emulsifying agents may be naturally-occuring gums such as gum acacia and gum tragacanth, naturally-occuring phosphatides such as soy bean and lecithin, esters or partial esters derived from fatty acids and hexitol anhydrides, for example, sorbitan monooleate, condensation products of said partial esters with ethylene oxide, for example polyoxyethylene sorbitan monooleate. The emulsions may also contain sweetening and flavoring agents. Syrups and elixirs may be formulated with sweetening agents, for example, glycerol, propylene glycol, sorbitol or sucrose. Such formulations may also contain a demulcent, a preservative and flavoring and coloring agents.

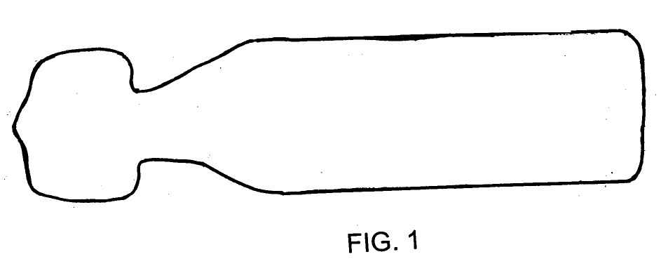

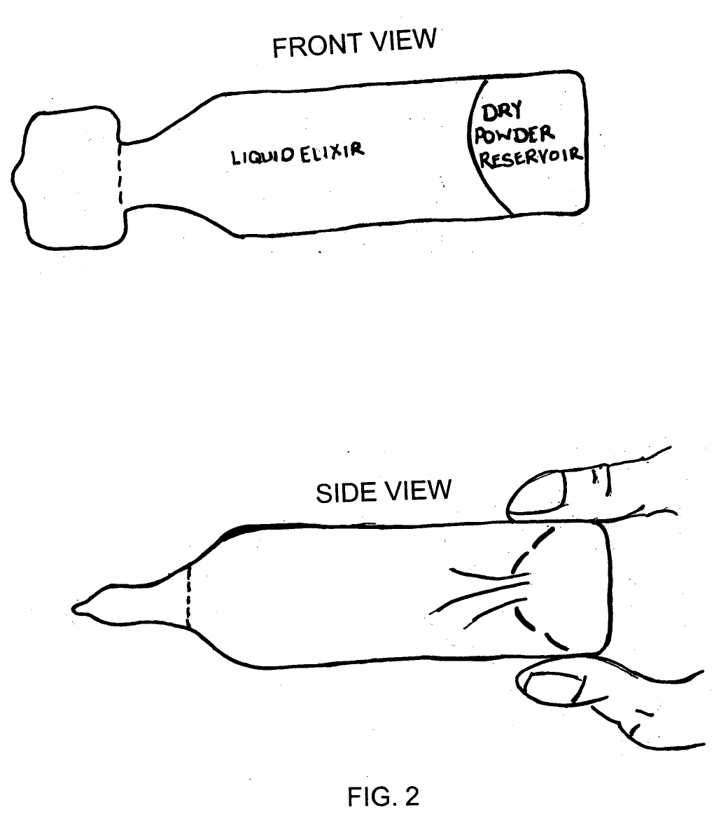

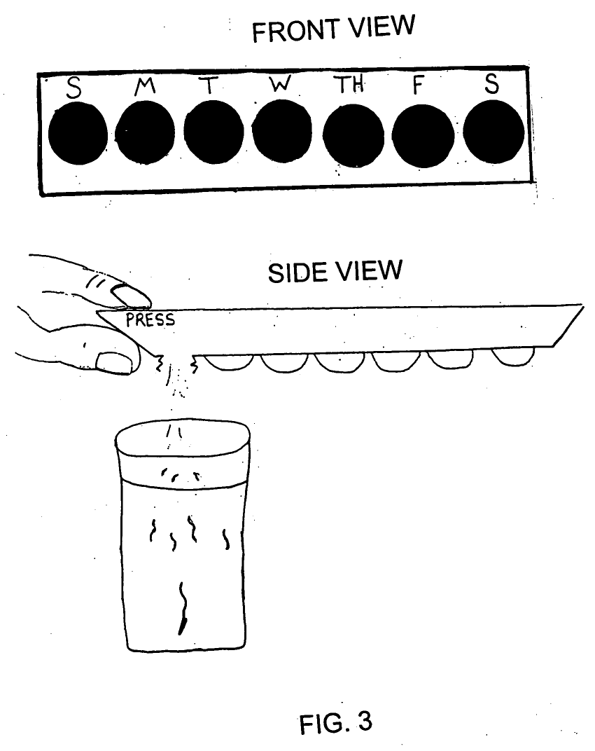

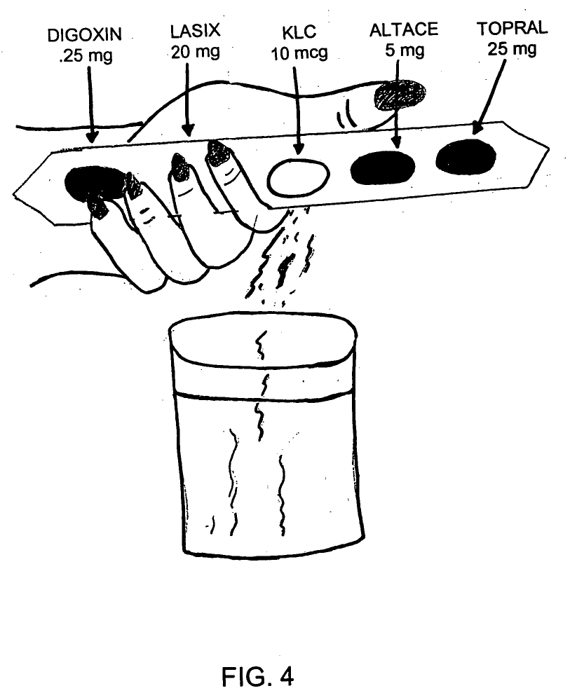

Pharmaceutical formulations of the invention are prepackaged in periodic (e.g., daily, twice-daily, four times a day) dosage units to simplify the drug-taking regimen of a patient, thereby increasing patient compliance. Formulations described herein that are daily dosage units, for example, are packaged such that the contents of a single prepackaged unit are consumed by a patient per day. The pharmaceutical formulations described herein can be packaged as periodic (e.g., daily, twice-daily, three times a day, four times a day) dosage units in a variety of ways. For example, packaging for a liquid or powder pharmaceutical formulation can take the packaging form of a plastic (e.g., disposable) receptacle (e.g., dispenser) with a twist-off cap (FIG. 1). When the pharmaceutical formulation is a dry powder that is combined with a liquid immediately before ingesting, the packaging can take the form of a plastic receptacle (e.g., dispenser) having a first compartment (e.g., dry plastic reservoir) for containing the dry powder and a second compartment for containing the liquid, the compartments separated from one another by a rupturable seal or barrier, such that rupturing of the seal or barrier (e.g., by squeezing, twisting, etc.) causes the powder and the liquid to mix (FIG. 2). As another example, a blister pack can be used for packaging pharmaceutical formulations in powder form, allowing for easy dispensing of the formulation from the blister pack into the patient's drink of choice (FIG. 3). Another example of a blister pack is shown in FIG. 4, in which each drug in powder form of a particular treatment regimen is contained within a separate compartment (i.e., blister) of the blister pack, each compartment containing the appropriate dose of each drug according to the treatment regimen. This embodiment is particularly useful for a combination of drugs that are not suitable for storage together in one compartment, as well as for a drug regimen in which one of the combination of drugs may be omitted at some point during the regimen. By pressing on each compartment (i.e., blister) of the blister pack with sufficient force, the powder is released, preferably into a liquid to form a dissolved or suspended drug mixture which the patient can then drink. In this embodiment, a patient consumes the contents of one such blister pack each day.

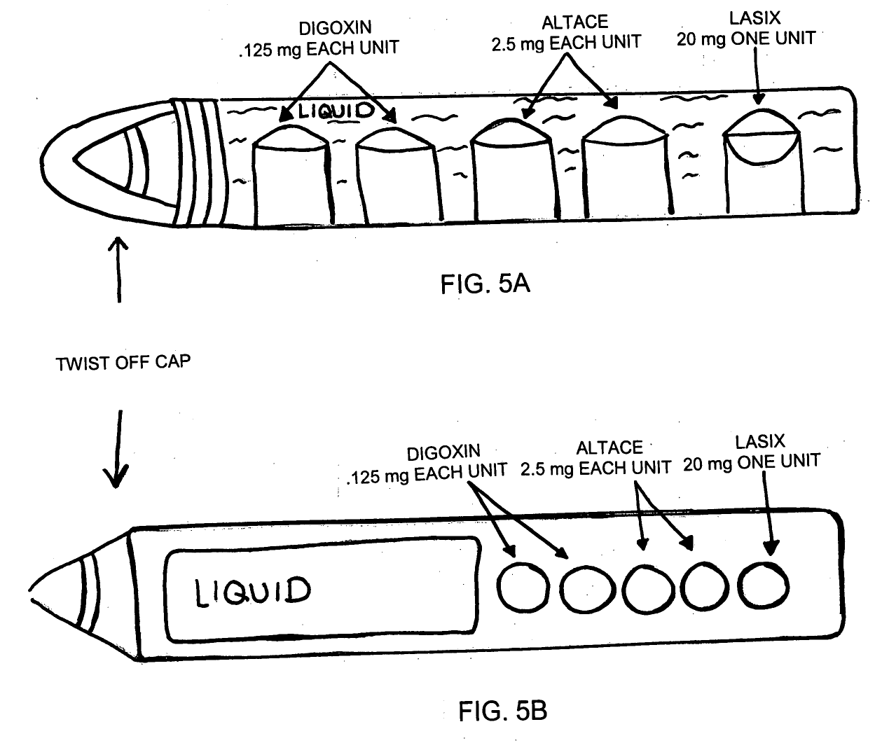

Additional forms of packaging suitable for use in the invention are those shown in FIGS. 5A and 5B. In each of these embodiments, a receptacle (e.g., dispenser) contains a liquid as well as multiple plastic or metallic dry deformable compartments adapted to accommodate pharmacologically active ingredients. Each compartment contains a pharmacologically active ingredient in powder form at a particular dose. In FIG. 5A, the receptacle contains five plastic or metallic dry deformable compartments, each containing a pharmacologically active ingredient in powder form. These compartments are surrounded by a liquid such that when the deformable compartments are ruptured (e.g., by squeezing), the powder is released from the compartments and mixed with the surrounding liquid. By shaking or tapping the receptacle, the patient (or person administering the formulation to the patient) mixes all of the pharmacologically active ingredients with the liquid, and can then twist-off the cap to either drink the pharmacologically active ingredient-containing liquid directly, or dispense the pharmacologically active ingredient-containing liquid into a glass of water, juice, or other desirable liquid for consuming. In FIG. 5B, the receptacle also contains five plastic or metallic dry deformable compartments, each containing a pharmacologically active ingredient in powder form, but instead of a liquid surrounding all of the compartments, the liquid in this receptacle is contained within its own compartment. To ingest the contents of the receptacle, a patient first ruptures the powder-containing compartments, releasing the powder, then ruptures the liquid compartment, thereby releasing liquid such that it contacts the powder. Alternatively, the patient can rupture the liquid-containing compartment before rupturing the powder-containing compartments. Again, by shaking or tapping the receptacle, the patient mixes all of the pharmacologically active ingredients with the liquid, and can then twist-off the cap to either drink the pharmacologically active ingredient-containing liquid directly, or dispense the pharmacologically active ingredient-containing liquid into a glass of water, juice, or other desirable liquid for consuming. The receptacles shown in FIGS. 1, 2, and 5 have twist-off caps, however, any suitable removable or rupturable cap may be used. In some embodiments, pharmaceutical formulations described herein may be packaged in child-proof or child-safe receptacles or dispensers.

### EXAMPLES

The present invention is further illustrated by the following specific examples. The examples are provided for illustration only and are not to be construed as limiting the scope or content of the invention in any way. Appropriate medications and dosages are known to those of skill in the art, and it is to be appreciated that different physicians practice medicine in different ways, and have varying preferences for different drugs.

### Example 1

### Pharmaceutical Formulations for Congestive Heart Failure

Standard regimens for congestive heart failure typically include a cardiac glycoside (e.g., digoxin, sold as Lanoxin™ by GlaxoSmithKline, Philadelphia, Pa.), a loop diuretic (e.g., furosemide, sold as Lasix™ by Aventis Pharmaceuticals, Bridgewater, N.J.), a potassium supplement or potassium sparing diuretic (e.g., spironolactone, sold as Aldactone™ by Pfizer, Inc., New York, N.Y.), an Ace inhibitor (e.g., ramipril, sold as Altace®, King Pharmaceuticals, Bristol, Tenn.) or an ATI receptor blocker for reducing blood pressure, a beta blocker (e.g., metoprolol succinate sold as Toprol™, AstraZeneca, Wilmington, Del.), or a combination of beta and alpha blocker (e.g., carvedilol, sold as Coreg™ by GlaxoSmithKliine, Philadelphia, Pa.). The dosages of each of these drugs and the selective addition or omission of one or more of them depends on the presence or absence of several fairly common clinical patient characteristics. Many patients with congestive heart failure secondary to either ischemic or non-ischemic dilated cardiomyopathy can be effectively treated with a standard daily regimen of 0.25 mg Digoxin, 20.0 mg, furosemide, 10.0 mg Potassium, 5.0 mg ramipril, and 25.0 mg metoprolol succinate. Instead of metoprolol succinate, carvedilol can be included in this regimen at one of many escalating twice-daily dosages to a maximum of 25.0 mg twice daily as tolerated. It is to be understood that a number of drugs are suitably interchangeable for inclusion in a pharmaceutical formulation for treating congestive heart failure according to the invention. For example, although ramipril is described above as an Ace inhibitor, any other suitable Ace inhibitor may be used. Many Ace inhibitors are known in the art, including benazepril, captopril, enalapril, fosinopril, imidapril, lisinopril, moexipril, quinapril, perindopril erbumine, and trandolapril. Similarly, a number of different beta blockers, diuretics, and cardiac glycosides exist and may be useful in the invention. Prescription and non-prescription drugs and their uses are described in Physician's Desk Reference, 60^{th} edition, Thomson PDR publishing, Montvale, N.J., 2006; and PDR: Guide to Drug Interactions, Side Effects, and Indications, 60^{th} edition, Thomson PDR publishing, Montvale, N.J., 2006.

Several fairly common clinical factors or co-morbid conditions require deviating from the standard regimen described above. Such conditions include, for example, impaired renal function, conduction system disease, diabetes, and bradycardia. For a patient having impaired renal function, the appropriate drug combination would have a higher diuretic dose and a lower dose of Ace inhibitor than the standard regimen described above, or the Ace inhibitor might be eliminated entirely from the regimen. An impaired renal function patient demonstrating a tendency toward relative hypotension would lead to a cautious reduction or elimination of the Ace inhibitor and possibly the beta blocker. Several contraindications for beta blockers exist, including asthma (obstructive or reactive airway disease), brittle diabetes, heart block greater than first degree, significant bradycardia, and claudication peripheral vascular disease. A beta blocker would likely be omitted from a pharmaceutical formulation for a patient having one of these contraindications. For those with low blood pressure, the Ace inhibitor dosage is reduced or eliminated. Greater diuretic dosages are provided in the combination for those who have such a need. For those with conduction system disease, bradycardia or renal disease, digoxin is omitted from the standard regimen above. For those patients having cardiomyopathy who are concomitantly taking statin drugs which may reduce antioxidants and therefore reduce heart function further, the dietary supplement Coenzyme-Q-10 may be added to the standard drug regimen.

Thus, although perhaps about six medications are considered when deciding on an appropriate drug formulation that would be effective for most patients with congestive heart failure and cardiomyopathy, the presence and dose of each drug in a regimen for a particular patient is adjusted after consideration of the patient's clinical factors (i.e., characteristics) and co-morbid conditions to yield the most effective, personalized treatment possible. For example, the digoxin dose within a particular regimen might be 0.25 mg for a standard regimen, 0.125 mg in a reduced regimen, or 0 mg in a regimen having no digoxin. Likewise for furosemide, a standard regimen might have 20.0 mg, an increased regimen might have 40.0 mg, and for some patients, furosemide might be eliminated entirely.

An example of a chart for use by a physician listing pharmacologically active ingredient combinations and daily dosages appropriate for treating congestive heart failure in patients having different clinical variables (characteristics) is shown below. As is well known in the medical arts, dosage for any one patient depends on many factors (as will be described in greater detail below), including the patient's size, body surface area, age, the particular medication to be administered, general health, and other pharmacologically active ingredients (e.g., drugs) administered concurrently.

| | | | | | | metoprolol | Coenzyme- |
|---|---|---|---|---|---|---|---|
| patient's | | digoxin | furosemide | Potassium | ramipril | succinate | Q-10 |
| condition | formulation | (mg) | (mg) | (mg) | (mg) | (mg) | (mg) |
| standard | I | .25 | 20.0 | 10.0 | 5.0 | 25.0 | +/− |
| renal | II | .125 | 40.0 | | 2.5 | 25.0 | |
| function | | | | | | | |
| impaired | | | | | | | |
| severely | III | | 40.0 | | | 25.0 | |
| impaired | | | | | | | |
| renal | | | | | | | |
| function | | | | | | | |
| low blood | IV | .25 | 20.0 | 10.0 | | | |
| pressure | | | | | | | |
| conduction | V | | 20.0 | 10.0 | 5.0 | +/−25.0 | |
| system | | | | | | | |
| disease | | | | | | | |
| bradycardia | VI | | 20.0 | 10.0 | 5.0 | | +/− |

Formulations I-VI described in the chart above can be formulated as prepackaged, pharmaceutical formulations packaged as periodic (e.g., daily, twice-daily, three times a day, four times a day) dosage units. For example, formulation I for treating congestive heart failure can be formulated as a liquid and packaged as daily dosage units in plastic receptacles such as the receptacle shown in FIG. 1. In this example, each day's dose (e.g., 0.25 mg digoxin, 20.0 mg furosemide, 10 mg potassium, 5.0 mg ramipril, 25.0 mg metoprolol succinate) is packaged in a single plastic receptacle, such that a 30 day prescription of the formulation includes 30 separate plastic receptacles, the contents of each to be consumed on a different day. In another embodiment, formulation I for treating congestive heart failure can be formulated as a powder and packaged as daily dosage units in a multi-day blister pack as shown in FIG. 3. In this embodiment, each blister of the blister pack contains 0.25 mg digoxin, 20.0 mg furosemide, 10 mg potassium, 5.0 mg ramipril, and 25.0 mg metoprolol succinate, the contents of one blister being consumed per day.

### Example 2

### Pharmaceutical Formulations for Coronary Artery Disease

Regimens for coronary artery disease typically include one or more of the following components: an aspirin, a 3-hydroxy-3-methylglutaryl coenzyme A reductase inhibitor (e.g., a statin), a B complex vitamin, a niacin, a nitrate (e.g., isosorbide dinitrate, pentaerythrityl tetranitrate, nitroglycerin) for inducing vasodilation and/or inhibiting platelet aggregation, a beta blocker, and a calcium channel blocker. Preferably, pharmaceutical formulations for coronary artery disease include a statin, a lipid-lowering agent, because studies of the impact of statins have shown that statin treatment consistently results in significantly lower cholesterol levels and dramatically reduces the incidence of coronary heart disease and vascular events such as heart attack and stroke. Several statins are known in the art, including pravastatin, lovastatin, simvastatin, fluvastatin, atorvastatin, and cerivastatin. In one embodiment, a pharmaceutical formulation of the invention for treating a patient having coronary artery disease includes aspirin, a statin, and a B complex vitamin in appropriate dosages. A pharmaceutical formulation of the invention for treating a patient having coronary artery disease can include any appropriate combination of aspirin, statin, B complex vitamin, niacin, nitrate, beta blocker, and calcium channel blocker. Which of these components are chosen for a formulation and the dosages that are chosen are based upon the particular patient's characteristics. For example, some coronary artery disease patients exhibit a number of side effects when taking nitrates including headaches and hypotension. Thus, for such a patient, a pharmaceutical formulation of the invention would include nitrate at a lower dosage than a pharmaceutical formulation for a patient not exhibiting headaches and hypotension in response to nitrates or in severe cases, the nitrate would be omitted. As another example, some coronary artery disease patients exhibit a number of side effects when taking calcium channel blockers, including headaches, constipation, and edema. A pharmaceutical formulation of the invention for such a patient would include a calcium channel blocker at a lower dose than a pharmaceutical formulation for a patient not exhibiting such side effects. Additionally, when choosing which calcium channel blocker to include in a pharmaceutical formulation, the desired effect on the patient's heart rate should be considered, as some calcium channel blockers slow the heart rate, while others do not. Because some calcium channel blockers are more compatible with beta blockers than other calcium channel blockers, the choice of calcium channel blocker may depend in part on whether a beta blocker is included in the formulation.

### Example 3

### Pharmaceutical Formulations and Chart for Treating HIV Patients

An example of a chart for use by a physician listing pharmacologically active ingredient combinations appropriate for treating HIV patients having different clinical variables (characteristics) is shown below. The chart below shows daily dosages, with emtricitabine/tenofovir, efavirenz, and simvastatin being administered once a day, and lopinavir/ritonavir being administered twice a day. Formulations I-II described in the chart below can be formulated as prepackaged, pharmaceutical formulations packaged as periodic (e.g., daily, twice-daily, three times a day, four times a day) dosage unit. For example, formulation I for treating a naïve HIV patient having a low CD-4 count can be formulated as a liquid and packaged as daily dosage units in plastic receptacles such as the receptacle shown in FIG. 1. In this example, each day's dose is packaged in a single plastic receptacle, such that a 30 day prescription of the formulation includes 30 separate plastic receptacles, the contents of each to be consumed on a different day. As another example, formulation II for treating a non-naïve HIV patient can be formulated as a liquid and packaged as daily dosage units in plastic receptacles such as the receptacle shown in FIG. 1. In this example, each day's dose is packaged in a single plastic receptacle, such that a 30 day prescription of the formulation includes 30 separate plastic receptacles, the contents of each to be consumed on a different day.

| | | | | lopinavir/ | |
|---|---|---|---|---|---|
| patient's | | emtricitabine, | | ritonavir | |
| condition | formulation | tenofovir | efavirenz | (twice a day) | simvastatin |
| naïve patient* | I | 200 mg | 600 mg | | |
| having a low | | emtricitabine, | | | |
| CD-4 count | | 300 mg | | | |
| | | tenofovir | | | |
| non-naïve** | II | 200 mg | | 300 mg | 20 mg |
| patient | | emtricitabine, | | lopinavir, | |
| | | 300 mg | | 150 mg | |
| | | tenofovir | | ritonavir | |

*Naïve patient - never having been treated for HIV before.
**Non-naïve patient - having been treated for HIV before.

### Example 4

### Pharmaceutical Formulations for Additional Conditions

Pharmaceutical formulations as described herein can be used to treat any number of diseases, disorders, or conditions. They can be used to treat a particular disease a patient is suffering from, or to mitigate side effects or symptoms related to the disease. For example, a pharmaceutical formulation for treating a cancer patient who suffers from weight loss or anorexia may include appetite stimulants. Such a formulation includes: prednisone at a dose of about 5 mg, twice a day; and megestrol acetate (e.g., Megace™, sold by Bristol-Myers Squibb, New York, N.Y.) at a dose of about 80 mg, twice a day. This formulation can additionally include methyl phenidate hydrochloride (e.g., Ritalin™, sold by Novartis, Basel, Switzerland) at a dose of about 5 mg, twice a day. In another example, a pharmaceutical formulation for treating a patient with mucositis includes diphenhydramine hydrochloride (e.g., Benadryl®, sold by Pfizer, Inc., New York, N.Y.) at a dose of about 25 mg, nystatin (e.g., Mycostatin®, sold by Bristol-Myers Squibb, New York, N.Y.) at a dose of about 5 cc, and viscous lidocaine at a dose of about 5 cc. An example of a pharmaceutical formulation for treating a patient suffering from pain includes oxycodone hydrochloride (e.g., Oxycontin®, sold by Purdue Pharmaceuticals, Stamford, Conn.) at a dose of about 10 mg, twice a day. An example of a chart for use by a physician listing drug combinations appropriate for treating cancer or HIV in patients having different clinical variables (characteristics) including appetite loss, pain, and trouble swallowing (e.g., mucositis) is shown below.

| | | | | methyl | | | | |
|---|---|---|---|---|---|---|---|---|
| patient's | | | megestrol | phenidate | oxycodone | diphenhydramine | | |
| condition | formulation | prednisone | acetate | hydrochloride | hydrochloride | hydrochloride | nystatin | lidocaine |
| cancer or | I | X | X | | | | | |
| HIV with | | | | | | | | |
| loss of | | | | | | | | |
| appetite | | | | | | | | |
| cancer or | II | X | X | X | | | | |
| HIV with | | | | | | | | |
| severe | | | | | | | | |
| case of | | | | | | | | |
| appetite | | | | | | | | |
| loss | | | | | | | | |
| cancer or | III | X | X | | X | | | |
| HIV with | | | | | | | | |
| loss of | | | | | | | | |
| appetite | | | | | | | | |
| and pain | | | | | | | | |
| cancer or | IV | | | | | X | X | X |
| HIV with | | | | | | | | |
| severe | | | | | | | | |
| mucositis | | | | | | | | |

While the above specification contains many specifics, these should not be construed as limitations on the scope of the invention, but rather as examples of preferred embodiments thereof. Many other variations are possible. For example, although the description of the invention focuses on pharmaceutical formulations for treating HIV/AIDS, cancer, and cardiac conditions, the invention could also be implemented in the treatment of numerous other conditions. As another example, nutraceuticals, dietary supplements, and vitamins can be included in a drug regimen involving a pharmaceutical formulation as described herein. As yet another example, two pharmaceutical formulations can be packaged in one dispenser. Accordingly, the scope of the invention should be determined not by the embodiments illustrated, but by the appended claims and their legal equivalents.

What is claimed is:

1. A pharmaceutical formulation delivery system, the system comprising:
a) a plurality of prepackaged pharmaceutical formulations each comprising at least two pharmacologically active ingredients in periodic dosage amounts for treating a condition,
each pharmaceutical formulation differing in active ingredients or dosage amounts from each other; and
b) a chart listing at least a first set of patient characteristics relating to the condition and a second set of patient characteristics relating to the condition, the chart indicating a pharmaceutical formulation of the plurality of pharmaceutical formulations that is suitable for treating a patient having the first set or second set of patient characteristics.

2. The pharmaceutical formulation delivery system of claim 1, wherein at least one pharmaceutical formulation of the plurality of pharmaceutical formulations comprises at least three pharmacologically active ingredients in periodic dosage amounts for treating a condition.

3. The pharmaceutical formulation delivery system of claim 1, wherein the prepackaged pharmaceutical formulations are in liquid form.

4. The pharmaceutical formulation delivery system of claim 1, wherein at least one pharmaceutical formulation of the plurality of prepackaged pharmaceutical formulations is in liquid form and is prepackaged in a plastic receptacle for containing a liquid, the receptacle comprising a twist-off cap.

5. The pharmaceutical formulation delivery system of claim 1, wherein at least one pharmaceutical formulation of the plurality of prepackaged pharmaceutical formulations comprises a powder and a liquid and is prepackaged in a plastic receptacle, the plastic receptacle comprising a twist-off cap, a first compartment for containing the powder, and a second compartment for containing the liquid, the first and second compartments separated by a rupturable seal such that rupturing of the seal exposes the powder to the liquid.

6. The pharmaceutical formulation delivery system of claim 1, wherein at least one pharmaceutical formulation of the plurality of pharmaceutical formulations is in powder form and is prepackaged in a sufficient amount to provide dosing for seven days, the at least one pharmaceutical formulation prepackaged in a blister pack including a plurality of deformable blisters therein, each deformable blister containing one day's dose of the at least one pharmaceutical formulation.

7. The pharmaceutical formulation delivery system of claim 1, wherein at least one pharmaceutical formulation of the plurality of pharmaceutical formulations is in powder form and comprises at least three pharmacologically active ingredients, the at least one prepackaged pharmaceutical formulation prepackaged in a blister pack including a plurality of deformable blisters therein, each deformable blister containing one pharmacologically active ingredient.

8. The pharmaceutical formulation delivery system of claim 1, wherein at least one pharmaceutical formulation of the plurality of prepackaged pharmaceutical formulations comprises a powder and a liquid and is prepackaged in a plastic receptacle, the plastic receptacle comprising a twist-off cap, at least a first and a second compartment for containing the powder, and a third compartment for containing the liquid, the at least first and second compartments separated from the third compartment by rupturable seals such that rupturing of the seals exposes the powder to the liquid.

9. A method of treating a patient having a particular condition, the method comprising the steps of:
a) evaluating the patient and determining a set of patient characteristics relating to the condition;
b) selecting a prepackaged pharmaceutical formulation from a chart listing a plurality of sets of patient characteristics relating to the condition and a plurality of prepackaged pharmaceutical formulations, each pharmaceutical formulation comprising at least two pharmacologically active ingredients in periodic dosage amounts for treating the condition, the chart indicating which of the plurality of pharmaceutical formulations is suitable for treating the patient; and
c) administering the prepackaged pharmaceutical formulation to the patient.

10. The method of claim 9, wherein the condition is selected from the group consisting of: HIV/AIDS, cancer, heart failure, and coronary artery disease.
